(19) United States
(12) Patent Application Publication
(10) Pub. No.: US 2025/0352792 A1
(43) Pub. Date: Nov. 20, 2025

(54) AN ELECTRICAL NERVE STIMULATION SYSTEM FOR STIMULATING ONE OR MORE NERVES OF A MAMMAL WITH AN ELECTRICAL SIGNAL

(71) Applicant: QMEDIC B.V., Bergen Op Zoom (NL)

(72) Inventors: Marinus Joannes Leonardus QUINTUS, Bergen Op Zoom (NL); Erik Jan DE JONG, Bergen Op Zoom (NL)

(21) Appl. No.: 18/866,640

(22) PCT Filed: May 16, 2023
(86) PCT No.: PCT/NL2023/050275

§ 371 Date: Nov. 18, 2024

(30) Foreign Application Priority Data
May 18, 2022 (NL) 2031921

(51) Int. Cl. A61N 1/36; G16H 40/63

(52) U.S. Cl. CPC A61N 1/36034; A61N 1/36021; A61N 1/36031; G16H 40/63

(57) ABSTRACT

The invention relates to an electrical nerve stimulation system for stimulating one or more nerves of a mammal with an electrical signal, said system comprising: a signal generation unit arranged for generating electrical stimulation pulses wherein an intensity of said pulses is configured for stimulating said one or more nerves of said mammal: a pair of electrodes connected to said signal generation unit and arranged for placement of said electrodes on the outer skin of said mammal: a control unit arranged for control of said signal generation unit: a power supply unit for powering said signal generation unit and said control unit: wherein said signal generation unit comprises an inductor for inducing said stimulation pulses applied in a symmetric inversed manner to said pair of electrodes, and wherein the duration and/or timing of said stimulation pulses are varied over time.

### TECHNICAL FIELD

The invention relates in general to electro stimulation of nerves of a mammal. More in particular, the invention relates to stimulation of nerves with an electrical nerve stimulation system in which an electrical signal is generated and applied to the mammal trough a pair of electrodes applied to the outside skin of the mammal.

### Background of the Invention

Nerve stimulation by applying an electric current to one or more of the nerves of a mammal is considered a typical and often used therapy for reduce pain. Acute but also chronic pain can be lowered, minimized or at least alleviated with such therapy.

Such nerve stimulation therapy is considered, in comparison with for example surgery, a simple, safe, relatively low cost and possibly self-administered type of therapy. Amongst those reasons, such therapy has become increasingly popular in recent years. Given the trend of the rapid increase in the number of patients experiencing pain, requiring medical care, and the rapid increase in health-care costs involved in treating these patients, the demand for such cost-efficient yet effective forms of therapy is increasing.

Nerve stimulation therapy may be used in a variety of application and for a variety of treatments, for example to alleviate pain for example in the lower back area. The nerve stimulation therapy may also be used to diagnose and correct disabilities to the musculoskeletal system. These applications are however merely examples and as the use of such therapy may also be effective for other problems relating to muscles, orthopaedic problems but mostly nerve related problems or disabilities of a human or other mammal.

Known nerve stimulation therapy is based on devices or systems which generate a low electrical current flowing through part of the body of the human from one electrode to the other. The effectiveness of the therapy is mostly based on the way in which the stimulation is applied to but also perceived by the nerves. Typically, the nerves will perceive the most stimulation from an intense signal. Such intense signal may be considered the most effective way of treatment of the disability or problem relating to the nerve of the human.

Such intense level of stimulation, however, is considered inherent painful for the patient. As a result, the patient will experience the therapy as unpleasant and may be ill-affected be by such form of therapy.

Moreover, it has been determined that increase of the level of intensity of the stimulation is non-linear as the steps of increasing in intensity may not result in similar increase in effectiveness of the treatment.

Further, performing the therapy by applying an intense level of stimulation on the nerves, may suppress the pain for which the patient is treated, but may not resolve the underlying cause of the pain and thus, the patient will require numerous sessions of treatment and may experience recurring pain when the treatment has ended.

And so there is a limit to the extent to which the patient on the one hand can still experience the treatment as pleasant or at least acceptable, and on the other hand a limit to the extent to which the effectiveness of the treatment increases under the increase in intensity of the signal. Also, current nerve stimulation therapies are considered mostly to suppress pain in stead resolving the pain or providing pain relief.

As such, it is an object of the present invention to provide a nerve stimulation system which is not experienced as painful, and the treatment is perceived as acceptable for the patient, while the effectiveness of the treatment is improved.

### SUMMARY OF THE INVENTION

According to a first aspect of the present disclosure, there is provided an electrical nerve stimulation system for stimulating one or more nerves of a mammal with an electrical signal, said system comprising:
a signal generation unit arranged for generating electrical stimulation pulses wherein an intensity of said pulses is configured for stimulating said one or more nerves of said mammal;
a pair of electrodes connected to said signal generation unit and arranged for placement of said electrodes on the outer skin of said mammal;
a control unit arranged for control of said signal generation unit;
a power supply unit for powering said signal generation unit and said control unit;
wherein said signal generation unit comprises an inductor for inducing said stimulation pulses applied in a symmetric inversed manner to said pair of electrodes, and said control unit further being arranged to control a variation of the duration and/or timing of said stimulation pulses.

The electrical nerve stimulation system consists of several components, amongst which a power supply to power each active component of the system, a pair of electrodes which are to be connected as electrode patches to the skin of the mammal, and a control unit and signal generation unit.

The signal generation unit and control unit are the core components of the system as with these components the signal that is supplied to the electrodes is generated and controlled thereby.

The control unit controls the operating of the signal generation and thus controls whether or not the signal is applied to the electrode pair, thereby starting the treatment of the mammal.

The signal generation unit generates the signal as electrical stimulation pulses. These pulses are configured for use in a nerve stimulation system which means that these differ from the pulses generated in known muscle stimulation devices. Muscle stimulation is performed with more continues signals as therapy of muscles is typically done with higher continues signals than those of nerve stimulation. Continues is in this context to be understood as the amplitude and frequency of the stimulation signal.

The invention is based on the insight that typical nerve stimulation can be inherent painful and is thus susceptible to improvement if the stimulation pulses are tuned to the behaviour of the nerves themselves instead of increasing the effectiveness of the treatment by increasing the intensity, which is more typical for known stimulation systems. Tuning according to the invention maximizes the stimulation effect and thereby the effectiveness of the treatment.

Known stimulation systems only suppress the pain by administering stimulation pulses to alleviate acute and chronic pain by reducing or suppressing the sensitivity of the body or more particularly the nerves and thereby the pain. The proposed system however tackles the root or cause of the pain in the nerves by applying the stimulation pulses in a symmetric inversed manner to the pair of electrodes. This way the ion channels in the nerves are prevented from building up a charge, by which the sensitivity of the nerves stays constant.

It has been found that the nerves of most mammals may be become accustomed to the administered stimulation pulses and as such, may thus learn from the pulses to set a bias by which the stimulation may become less effective over time, and that this can be prevented or at least minimized by arranging a variation in the stimulation pulse such that the time duration of the pulses and/or the timing of each pulse is varied to a certain degree, which variation is arranged under control of the control unit. The level of variation preferrable is fixed such that for example the level of variation of the time duration is for example in the range of a factor 2, 5, 10, 20, 25, 50, or 100, and similarly for the timing which preferably has a smaller range of variation of for example 1.1, 1.2, 1.5, 1.8, 2, 3, 5 or 10.

The variation in duration and or time interval, may be further extended with a variation in frequency and less preferably intensity of the signal. It has been found that such variation is effective in resetting the nerve system and thereby increasing the level of effectiveness of the treatment without increasing the dose or intensity and thus suppressing the pain or causing an inherent painful treatment for the patient.

In an example, wherein the signal generation unit comprises a five-pole inductor or a more than five-pole inductor, such as a seven-pole inductor, a 9 pole-inductor, an eleven pole inductor or any other higher pole inductor, preferably having odd number of poles, for generating the stimulation pulses.

With a five (or more)-pole inductor the signal generation unit is able to generate stimulation pulse in an effective manner. Such an inductor also allows to create symmetric inversed stimulation pulses which has been found to be beneficial and effective for the treatment.

In an example, the control unit is arranged to control generation of the stimulation pulses as symmetric inversed stimulation pulses.

It has been found that typical therapy systems are less effective as compared to symmetric inversed bipolar stimulation as the ion channels in the nerves to not build-up as a charge, by which the sensitivity of the nerves stays constant, to enable the selective stimulation on a constant level, enable minimized stimulation voltages and currents for continued stimulation, and enable continues effectiveness of the stimulation being performed.

In an example, the control unit is arranged to measure a nerve stimulus response, and wherein the signal generation unit is arranged to adapt the stimulation pulses based on the measured stimulus response.

In an example, the measured nerve stimulus response, is measured both positively and negatively, hence, both a positive response and a negative response is measured by the system.

In an example, the adapting comprises varying the time duration of the stimulation pulses.

In an example, measuring the nerve stimulus response, comprises measuring a change of frequency between the generated stimulation pulse and the measured nerve stimulus response.

In an example, the control unit is arranged to operate in a scanning mode and a operational mode, wherein in the scanning mode, the control unit is arranged to generate a sequence of scanning stimulation pulses, and measuring a sequence of corresponding nerve stimulus responses, and wherein in the operational mode, the control unit is arranged to generate a sequence of operational stimulation pulses. Preferably, the sequence of operational stimulation pulses comprise more extensive stimulation pulses than the calibration stimulation pulses, such that more extensive stimulation pulse of the operation mode have a wider range of variety of timing and/or time-duration than the scanning stimulation pulses.

In an example, the control unit is arranged to generate a first and second sequence of scanning stimulation pulses, wherein the first sequence comprises a larger number of scans then the second sequence.

In an example, the varying of the time duration of the stimulation pulses comprises randomly increasing or decreasing the time duration of each stimulation pulse within a predefined time duration bandwidth.

Random may in accordance with the present disclosure be interpreted as having a dynamically or varying frequency and/or timing, time-duration, repetition rate of a sequence of pulses, etc.

In an example, an intensity of the stimulation pulses is configurable.

Although the stimulation pulses may have a lower energy contents as compared to stimulation pulses used in muscle treatment, but also compared to typical nerve treatment, the energy contents of the pulses according to the present disclosure, may in an example, be configurable. The configuration may be set in a discrete manner, or a continuous manner and may be set prior to the treatment or may be changed over the course of the treatment.

In an example, the configuration of the intensity of the stimulation pulses is adapted during treatment in accordance with a predefined stimulation pattern.

In an example, the signal generation unit is arranged for generating the stimulation pulses as symmetric stimulation pulses wherein the pulses supplied to each electrode of the pair of electrodes is inverted in respect of each other.

In an example, the variation of the stimulation pulses are varied in accordance with a randomized variation pattern, which variation pattern is preferably a recurring variation pattern.

In an example, the randomized pattern comprises a pattern of stimulation pulses in which at least one or more is varied of the group of pulse duration, pulse frequency, duty-cycle, time-interval.

Selecting a certain stimulation pattern may be done in accordance with a difference in pulse width modulation of the signal so stimulation levels can be accurately controlled. The pulse width modulation may further preferably be controlled based on a measured nerve response

The nerve response may be measured based on measuring the actual nerve response to a small stimulus which determines the location where the nerve system shows a local stable position.

In an example, the stimulation signal may be tuned trough a hardware component such as the signal generation unit, which may be arranged for inductive and capacitive signal properties which enable the nerves to be dominant in the response and impact of the stimulation

To this end, a five-pole low induction inductor may be used to stimulate the nerves with minimum load, so the response is determined by the introduced energy and the nerves themselves. The minim load stimulations may result in the lowest pain response while enabling stimulation of the clinically relevant nerves. Neuro-feedback training may be minimized by applying fast changing signals that changes too fast for the nerve system to learn or adapt. For nerve systems that are locked in a local stable position this can result in a reset after which the best stable position can be redetermined. The duration, frequency and/or time interval variations may be used to reset the nerve system according to an example of the present disclosure. Similarly, a more than five pole inductor, e.g. a seven-pole inductor, a 9 pole-inductor, an eleven pole inductor or any other higher pole inductor, preferably having odd number of poles, are applicable as well.

The modulation level may further be controlled based on pulse width modulation of the primary signal of the five or more-pole inductor.

In an example, the system comprising an interface unit arranged to provide an operator of the system to control the control unit.

In an example, the control unit comprises a memory unit storing a plurality of predefined stimulation signal varying time duration patterns, for the control unit to select one of the patterns and to control the signal generation unit to induce the stimulation pulses in accordance with the selected pattern.

In an example, the control unit comprises a memory unit storing a plurality of predefined stimulation signal intensity patterns, for the control unit to select one of the patterns and to control the signal generation unit to induce the stimulation pulses in accordance with the selected pattern.

The invention will now be explained in more detail with reference to the appended FIGURES, which merely serve by way of illustration of the invention and which may not be construed as being limitative thereto.

### BRIEF DESCRIPTION OF THE DRAWING

FIG. 1 shows, in a schematic form, shows an embodiment of a system according to an aspect of the present invention.

### DETAILED DESCRIPTION

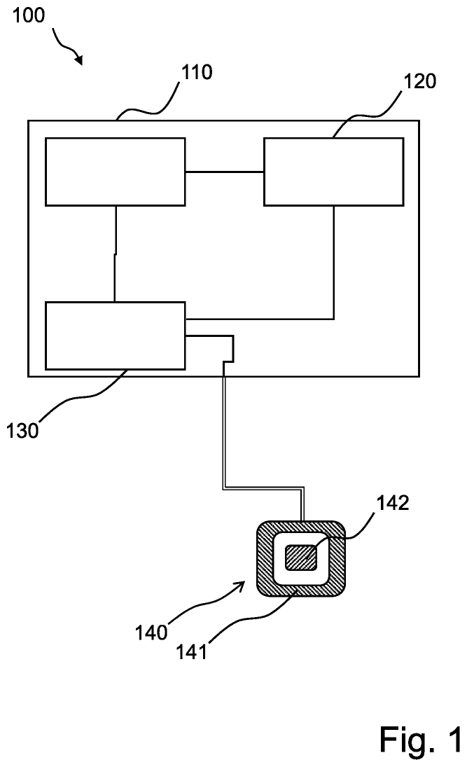

FIG. 1 shows a electrical nerve stimulation system 100 for stimulating one or more nerves of a mammal with an electrical signal.

The system 100 comprises several components of which the core components are shown in FIG. 1. It is expressed that the system may comprises several additional components which are not shown in the FIGURE, and the skilled person will appreciate which components those are.

The system 100 at least comprises a signal generation unit 110. The signal generation unit is arranged or configured for generating the electrical stimulation pulses of the system and is thus a signal generator which is arranged to generate predefined signal patterns and/or generate signal patterns with a certain level of variation, especially in the time domain, i.e. varying in timing of the start of subsequent pulses and the time duration of each pulse. The pulses may be bipolar pulses or pulses having a certain shape such as sine, square, sawtooth or triangle shaped pulses. The pulses may however also be modulation according to a certain combination of signals such that a complex signal is generated.

The signal generation unit 110 generates the signals which are eventually administered to the mammal or more particularly patient or human. Throughout the description mammal, patient and human may be used as examples of the subject to which the treatment is administered. As such, for any reference to patient or human, any other type of mammal may also apply.

The signal generation unit 110 generates the signals at a certain maximum energy level which is preferably limited by 500 mJoule but more preferably by 300 mJoule, measured on a load of 500 Ohm. The pulses may vary in length but are preferably maximized by a length of 0.5 seconds of time duration and more preferably 0.3 or 0.1 second. The pulses generated by the signal generation unit 110 may further be limited to voltage amplitude of 500 Volt peak value, measured under open-circuit condition, thus without load. More preferably however the output voltage is limited to 350 Volt, e.g. by use voltage clamping means such as for example by use of two Zener diodes.

The signal generation unit 110 is controlled by a control unit 130, the control unit may comprise a computing unit such as a general purpose computing unit, or a dedicated computing unit. Preferably, the control unit 130 may comprises a low-power microcontroller. This has the advantage that the unit is low-power, easily configurable, highly compatible and also accessible and thereby configurable through I/O interfaces such as different interfaces. Preferably, the signal generation unit 110 comprises a microcontroller or central processing unit with several cores, which allows to separate operational tasks and dedicate certain cores to for example control of the signal generation and a separate core for communication and control of the device or user operation.

The system 100 further comprises a power supply unit 120 to power the signal generation unit 110 and the control unit 130. The power supply may be configured to supply Direct Current, DC, power to the control unit, and to signal generation unit. The power supply unit 120 is in the example shown in FIG. 1 illustrated to be incorporated into the system and thus the housing of the stimulation device 100. This is however merely an example as the power supply unit 120 may also be an external power supply which is arranged to convert AC-mains into DC power for powering the units 110 and 130 of the device 100. These however may be further changed by increasing or decreasing the DC voltage, e.g. by a buck, a boost, or a buck-boost DC-DC converter. Preferably the signal generator is configured to limited the output current or input current such that the maximum energy administered to the patient to not exceed the 300 mJoule limit.

The power supply unit 120 may also comprises a battery unit which may be configured either as an auxiliary, backup power supply in case of absent or failing AC mains, or may be configured as a primary power supply in permanent absence of AC main. Preferably, in case of a battery unit as auxiliary power supply, the battery unit may be charged by the primary power supply from the AC-DC converter.

The system 100 further comprises a pair of electrodes 140 or stimulation electrode 140. The FIGURE shows one electrode pair integrated into a single electrode pad or probe 140. The pad 140 thus has at least two electrodes, 141, and 142 to apply the stimulation signal. The electrodes are coaxially positioned with respect to each other and configured in an outer ring and inner ring. The electrodes may comprises a highly electrically conductive surface such a metallic layer.

The system 100 is, as indicated, a simplistic illustration of the system according to the present disclosure and may comprise additional components. The power supply unit 120 may comprises a separate AC-DC converter and a separate D-DC converter to generate one or preferably several voltage levels and charge voltage for an auxiliary battery unit.

The signal generation unit 110 may be arranged to comprise a energy limiting circuit to limit the maximum power or energy which is administered thought the electrodes to the patient. The signal generation unit 110 may further comprise a debug interface such that direct debugging of the microcontroller is enabled. The signal generation unit also may comprise an overvoltage protection to protect the maximum voltage level that his administered to the patient. The control unit further may comprise a watchdog to monitor the cores of the microcontroller or general purpose computing unit and when one of the cores is unresponsive, reset the controller which may disable the output channel to the electrode. If the pulse generating unit would continue to generate pulses, they would in such case not be connected to the electrode. The overvoltage protection is preferably connected to the channel enable such that the simulation pulses may be limited by a disable signal applied to the channel enable circuit. Each of the aforementioned units may be connected to the microcontroller through an interface such as a the GPIO interface. The control unit may further be connected to a display, control input means and status indication LED's. By which for example stimulation patterns may be selected, operational status may be indicated or intensity levels may be changed.

The core of the system is the signal generation unit 110 which may comprises a charge coil or inductor, in particular a 5, 7, 9, 11 or more pole inductor. And a measuring circuit to measure the complex impedance of the tissue of the patient when the stimulation pulses are administered through the electrodes. In an example, the measuring circuit may be arranged to measure the frequency change of shift of the nerve stimulus response, e.g. measuring a change of frequency between the generated stimulation pulse and the measured nerve stimulus response.

The inductor of the signal generation unit 110 is arranged for inducting the stimulation pulses in a symmetric inversed manner to the electrodes 141, 142, wherein the duration or timing and preferably both the duration and the timing of the stimulation pulses are varied over time.

The signal generation unit 110 comprises an inductance which may comprise a multi-coil based inductor or driver unit arranged for generating sequences of pulses. The pulses are generated under control of the control unit 110 and may for example comprise a PWM unit which generates exact timed pulses without software dependence. The output of the unit is passed through the Energy Limiter, which drives the pulse generation circuit or unit. It may consists of 1 coil with three symmetrical junctions of which the mid-pointy is connected to the power. When one of the two other connections is connected to GND via a transistor that part of the coil is charged. When the connection is broken the stored energy in the coil will be released through the electrodes in either a positive or negative pulse depending on which side of the coil was charged. The circuit is preferably connected to a DC voltage and a diode may be connected to each microcontroller output to limit the possible negative spikes at the output coming from the transistor and coil. Zener diodes may be connected to each collector of a Darlington to limit the collector voltage to an acceptable level at the moment it is switched off. The cut-off voltage is preferably set at any value below 140V. The coil is preferably a ferrite core based coil having 4 wiring sections of each 150 windings and hence a five-pole connecting terminal configuration.

Similarly, a more than five pole inductor, e.g. a seven-pole inductor, a nine-pole-inductor, an eleven-pole inductor or any other higher pole inductor, preferably having odd number of poles, are applicable as well, and may preferably have a ferrite core, with x−1 number of winding sections for a respective x-number pole inductor. For example, a seven pole inductor may have six winding sections, which may have, by example, have similar or different number of windings, i.e. 150.

Preferably, the system further comprises a damping circuit which is connected between both electrodes and comprises two capacitors in series, followed by two resistors in parallel. The damping levels may be configurable by incorporating switching means on each of the resistors such that, based on the ratio between the resistors, for example a zero, low, medium or high dampening can be selected.

The measuring circuit preferably only measures on one electrode, e.g. the positive electrode. The voltage of the electrode is reduced to CPU or microcontroller-safe levels, after which the analogue signal is converted to digital signal. The signal may then fed to the CPU or microcontroller which measures the timing of the signal using an Input Capture Module or ADC or similar and based on the results decides whether or not the electrode is making skin contact.

Those skilled in the art will appreciate that the system according to the present disclosure may be manufactured by other methods than disclosed above and that the embodiments and aspects described are merely examples whereas the scope of protection is defined by the appending claims.

1. An electrical nerve stimulation system for stimulating one or more nerves of a mammal with an electrical signal, said system comprising:
a signal generation unit arranged for generating electrical stimulation pulses wherein an intensity of said pulses is configured for stimulating said one or more nerves of said mammal;
a pair of electrodes connected to said signal generation unit and arranged for placement of said electrodes on the outer skin of said mammal;
a control unit arranged for control of said signal generation unit;
a power supply unit for powering said signal generation unit and said control unit;
wherein said signal generation unit comprises an inductor for inducing said stimulation pulses applied in a symmetric inversed manner to said pair of electrodes, and said control unit further being arranged to control a variation of the duration and/or timing of said stimulation pulses.

2. The nerve stimulation system according to claim 1, wherein said signal generation unit comprises an inductor for generating said stimulation pulses having at least five-poles.

3. The nerve stimulation system according to claim 1, wherein said inductor has or of five-poles, seven-poles, nine-poles or eleven poles.

4. The nerve stimulation system according to claim 1, wherein said control unit is arranged to control generation of said stimulation pulses as symmetric inversed stimulation pulses.

5. The nerve stimulation system according to claim 1, wherein said control unit is arranged to measure a nerve stimulus response, and wherein said signal generation unit is arranged to adapt said stimulation pulses based on said measured stimulus response, and preferably, the measured nerve stimulus response, is measured both positively and negatively.

6. The nerve stimulation system according to claim 5, wherein said adapting comprises varying said time duration of said stimulation pulses.

7. The nerve stimulation system according to claim 5, wherein measuring said nerve stimulus response, comprises measuring a change of frequency between the generated stimulation pulse and the measured nerve stimulus response.

8. The nerve stimulation system according to claim 5, wherein said control unit is arranged to operate in a scanning mode and a operational mode, wherein in said scanning mode, said control unit is arranged to generate a sequence of scanning stimulation pulses, and measuring a sequence of corresponding nerve stimulus responses, and wherein in said operational mode, said control unit is arranged to generate a sequence of operational stimulation pulses, wherein said sequence of operational stimulation pulses comprise more extensive stimulation pulses than said calibration stimulation pulses, and wherein said more extensive stimulation pulse of said operation mode in particular have a wider range of variety of timing and/or time-duration than said scanning stimulation pulses.

9. The nerve stimulation system according to claim 8, wherein said control unit is arranged to generate a first and second a sequence of calibration stimulation pulses, wherein said first sequence comprises a larger number of pulses then said second sequence, and wherein said first sequence comprises shorter pulse widths than said second sequence.

10. The nerve stimulation system according to claim 1, wherein said varying of said time duration of said stimulation pulses comprises randomly increasing or decreasing the time duration of each stimulation pulse within a predefined time duration bandwidth.

11. The nerve stimulation system according to claim 1, wherein an intensity of said stimulation pulses is configurable.

12. The nerve stimulation system according to claim 11, wherein said configuration of said intensity of said stimulation pulses is adapted during treatment in accordance with a predefined stimulation pattern.

13. The nerve stimulation system according to claim 1, wherein said signal generation unit is arranged for generating said stimulation pulses as symmetric stimulation pulses wherein the pulses supplied to each electrode of said pair of electrodes is inverted in respect of each other.

14. The nerve stimulation system according to claim 1, wherein said variation of said stimulation pulses are varied in accordance with a randomized variation pattern, which variation pattern is preferably a recurring variation pattern.

15. The nerve stimulation system according to claim 4, wherein said randomized pattern comprises a pattern of stimulation pulses in which at least one or more is varied of the group of pulse duration, pulse frequency, duty-cycle, time-interval.

16. The nerve stimulation system according to claim 1, further comprising an interface unit arranged to provide an operator of said system to control said control unit.

17. The nerve stimulation system according to claim 1, wherein said control unit comprises a memory unit storing a plurality of predefined stimulation signal varying time duration patterns, for said control unit to select one of said patterns and to control said signal generation unit to induce said stimulation pulses in accordance with said selected pattern.

18. The nerve stimulation system according to claim 1, wherein said control unit comprises a memory unit storing a plurality of predefined stimulation signal intensity patterns, for said control unit to select one of said patterns and to control said signal generation unit to induce said stimulation pulses in accordance with said selected pattern.
